(19) United States
(12) Patent Application Publication
(10) Pub. No.: US 2009/0048157 A1
(43) Pub. Date: Feb. 19, 2009

(54) USE OF ORGANIC COMPOUNDS

(76) Inventors: Moise Azria, Basel (CH); Claus Christiansen, Ballerup (DK)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/577,127

(22) PCT Filed: Oct. 10, 2005
(86) PCT No.: PCT/EP05/10892

§ 371 Date: Apr. 12, 2007

(30) Foreign Application Priority Data
Oct. 12, 2004 (GB) 0422644.5

(51) Int. Cl. A61K 38/23; A61P 19/10

(52) U.S. Cl. 514/12

(57) ABSTRACT

This invention relates generally to calcitonin and their use in bone growth. Specifically, the invention relates to the use of calcitonin, e.g. salmon calictonin, to stimulate new bone formation in patients in need thereof.

This invention relates generally to calcitonin and their use in bone growth. Specifically, the invention relates to the use of calcitonin, e.g. salmon calictonin, to stimulate new bone formation in patients in need thereof.

Calcitonins, e.g. salmon, (Asu-1, 7) eel or human calcitonin, of the invention are compounds which are long-chain polypeptide hormones secreted by the parafollicular cells of the thyroid gland in mammals and by the ultimobranchial gland of birds and fish. Calcitonin is mainly known as a potent inhibitor of osteoclastic bone resorption, which implicates bone attachment of osteoclasts and enzymatic degradation. For example, randomized clinical studies indicate that prolonged administration of salmon calcitonin (sCT) prevents bone loss, increase spine (trabecular) bone mass, and decreases fracture risk in postmenopausal woman (Chesnut C H et al., 2000, PROOF Study Group. Am J Med 109: 267-76; Trovas G P et al., 2002, J Bone Miner Res 17: 521-7; Reginster J Y et al., 1994, Eur J Clin Invest 24: 565-9; Overgaard K et al., 1992, BMJ 305: 556-61; Overgaard K, 1994, Calcif Tissue Int 55: 82-86).

The skeleton is constantly being remodeled by a balance between osteoblasts that lay down new bone and osteoclasts that break down, or resorb bone. In some disease conditions and advancing age the balance between bone formation and resorption is disrupted; bone is removed at a faster rate. Such a prolonged imbalance of resorption over a long duration leads to weaker bone structure and a higher risk of fractures.

In accordance with the present invention, it has now surprisingly been found that calcitonins, e.g. salmon calcitonin, exert overall bone forming effects in a clinical study (see Example 1) as only bone resorption markers are inhibited and bone forming markers are left unchanged. This finding is in contrast to a class of compounds, called bisphophonates, which lower the plasma level of bone resporption as well as bone forming markers (Gamero P et al., Markers of bone resorption predict hip fracture in elderly women: The EPIDOS study. J. Bone Miner. Res. 1996, 11 (10): 1531-38).

Thus, calcitonins, e.g. salmon calcitonin, are particularly useful in the treatment of a severe form of various bone loss disorders, including e.g. osteoporosis, osteopenia, tumors (especially tumor invasion and bone metastases (BM)), tumor-induced hypercalcemia (TIH) and multiple myeloma (MM).

Accordingly, the present invention provides a method for the treatment of a severe form of bone loss diseases in a patient in need of such treatment, which comprises administering an effective amount of a calcitonin, e.g. salmon calcitonin, to the patient.

Accordingly, the present invention provides a method to stimulate new bone formation in patients in need thereof, which comprises administering an effective amount of a calcitonin, e.g. salmon calcitonin, to the patient.

The invention further provides the use of a calcitonin, e.g. salmon calcitonin, in the preparation of a medicament for stimulation of new bone formation in mammals, e.g. humans.

The invention further provides the use of a calcitonin, e.g. salmon calcitonin, in the preparation of a medicament for the treatment of a severe form of bone loss diseases in mammals, e.g. humans.
The invention yet further provides the use of a calcitonin, e.g. salmon calcitonin, in the treatment of a severe form of bone loss diseases in mammals, e.g. humans.
The invention yet further provides the use of a calcitonin, e.g. salmon calcitonin, in the stimulation of new bone formation in mammals, e.g. humans.

Preferably the invention is used for the treatment of diseases and medical conditions in which calcitonin, e.g. salmon calcitonin, are used to have an overall bone growth effect. For example, the invention may be used for the treatment of diseases and conditions which involve excessive or inappropriate bone loss e.g. as the result of inappropriate bone metabolism. Examples of such diseases and conditions include severe forms of benign diseases and conditions such as osteoporosis of various genesis, periodontal disease; and especially malignant diseases such as MM and TIH and BM associated with various cancers, e.g. cancer of the breast, prostate, lung, kidney, ovary, or osteosarcoma. Generally the Invention may be used to treat severe bone loss diseases also in other circumstances where calcitonin, e.g. salmon calcitonin, may be used, e.g. when calcitonin, e.g. salmon calcitonin, are used in bone fracture healing, osteonecrosis or treatment of prosthesis loosening. Calcitonin, e.g. salmon calcitonin, are particularly useful for treating severe forms of diseases of bone metabolism including osteoporosis, osteoarthritis, and other inflammatory arthritides, and bone loss in general, including age-related bone loss, and in particular periodontal disease.

Thus, the invention relates to the use of calcitonin, e.g. salmon calcitonin, for the manufacture of a medicament for reducing the risk of bone fracture, preferably spinal and femoral bone fracture, in mammals, preferably a mammal, e.g. human, more preferably a post menopausal woman at risk of or having osteoporosis, e.g. severe osteoporosis. The medicament can be employed to increase stiffness and/or toughness at a site of a potential trauma or at a site of an actual trauma. Trauma generally includes fracture, surgical trauma, Joint replacement, orthopaedic procedures, and the like. Increasing bone toughness and/or stiffness generally includes increasing mineral density of particular bones, e.g. the subperiosteal site of the vertebrae and long bones, increasing strength of bone, and the like. Reducing incidence of fracture generally includes reducing the likelihood or actual incidence of fracture for a subject compared to an untreated control population. Moreover, femoral bone mineral density can predict the long-term risk for bone fracture in general (Melton et al, J. of Bone and Miner Res, 2003; 18 (2):312-318).

The uses and methods of the present invention represent an improvement to existing therapy of bone loss diseases in which e.g. bisphosphonates are used to prevent or inhibit development of bone metastases or excessive bone resorption, and also for the therapy of inflammatory diseases such as rheumatoid arthritis and osteoarthritis, as well as for all forms of osteoporosis and osteopenia.

Thus in the present description the terms “treatment” or “treat” refer to both prophylactic or preventative treatment as well as curative or treatment of severe bone loss diseases, in particular treatment of severe osteoporosis.

Thus in particular embodiments the invention provides: a method for the treatment of a severe form of bone loss disease in a patient in need of such treatment which comprises administering an effective amount of a calcitonin, e.g. salmon calcitonin, to the patient; the use of a calcitonin, e.g. salmon calcitonin, in the preparation of a medicament for the treatment of a severe form or severe forms of bone loss diseases; or the use of a calcitonin, e.g. salmon calcitonin, as an agent for treatment of a severe form or severe forms of bone loss diseases.

For these indications, the appropriate dosage will, of course, vary depending upon, for example, the particular calcitonin, e.g. salmon calcitonin, to be employed, the host, the mode of administration and the nature and severity of the condition being treated. However, in general, satisfactory results in animals are indicated to be obtained at a daily dosage from about 0.001 to about 0.1 mg/kg animal body weight. In larger mammals, for example humans, an indicated daily dosage is in the range from about 0.01 to about 10 mg of a compound according to the invention, conveniently administered, for example, in divided doses up to four times a day. The calcitonin, e.g. salmon calcitonin, may be administered in any usual manner, e.g. orally, for example in the form of tablets or capsules, or parenterally, for example in the form of injection solutions or solutions.

In another aspect, the invention provides the use of an orally administered pharmaceutical composition for the manufacture of a medicament for the treatment of osteoporosis and/or severe osteoporosis, said composition comprising between 0.1 and 3 mg of salmon calcitonin. More preferred are pharmaceutical compositions comprising less than 2.5 mg of Compound A, e.g. comprising between 0.1 and 2.5 mg, preferably comprising 0.15 mg, 0.4 mg, 0.8 mg, 1.0 mg, or 2.5 mg of Compound A. Even more preferred are pharmaceutical compositions comprising between 0.4 mg and 1 mg of Compound A, e.g. 0.8 mg of Compound A. Alternatively, the invention provides a method for the treatment of osteoporosis and/or severe osteoporosis in a patient in need of such a treatment which comprises administering between 0.1 and 3 mg of a calcitonin, e.g. salmon calcitonin, to the patient. More preferred method of treatment are treatments comprising less than 2.5 mg of Compound A, e.g. comprising between 0.1 and 2.5 mg, preferably comprising 0.15 mg, 0.4 mg, 0.8 mg, 1.0 mg, or 2.5 mg of Compound A. Even more preferred are treatments comprising between 0.4 mg and 1 mg of Compound A, e.g. 0.8 mg of Compound A.

The present invention also provides pharmaceutical compositions comprising the calcitonin, e.g. salmon calcitonin, in association with at least one pharmaceutical carrier or diluent for use in the treatment of a severe form of bone loss diseases. Such compositions may be manufactured in conventional manner. Unit dosage forms may contain for example from about 0.1 to about 3 mg, preferably from 0.4 to 1.0 mg, of the calcitonin, e.g. salmon calcitonin.

The oral delivery of calcitonins, e.g. salmon calcitonin, is generally the delivery route of choice since it is convenient, relatively easy and generally painless, resulting in greater patient compliance relative to other modes of delivery. However, biological, chemical and physical barriers such as varying pH in the gastrointestinal tract, powerful digestive enzymes, and active agent impermeable gastrointestinal membranes, makes oral delivery of calcitonins, e.g. salmon calcitonin, to mammals problematic, e.g. the oral delivery of calcitonins, which are long-chain polypeptide hormones secreted by the parafollicular cells of the thyroid gland in mammals and by the ultimobranchial gland of birds and fish, has proven difficult due, at least in part, to the insufficient stability of calcitonin in the gastrointestinal tract as well as the inability of calcitonin to be readily transported through the intestinal walls into the blood stream.

U.S. Pat. Nos. 5,773,647 and 5,866,536 describe compositions for the oral delivery of active agents, such as heparin and calcitonin, with modified amino acids, such as, N-(5-chlorosalicyloyl)-8aminocaprylic acid (5-CNAC), N-(10-[2-hydroxybenzoyl]aminodecanoic acid (SNAD), and N-(8-[2-hydroxybenzoyl]amino)caprylic acid (SNAC). In addition, WO 00/059863 discloses the disodium salts of formula I

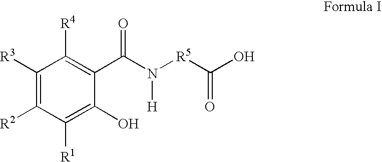

wherein

R¹, R², R³, and R⁴ are independently hydrogen, —OH, —NR⁶R⁷, halogen, C₁-C₄alkyl, or C₁-C₄alkoxy;

R⁵ is a substituted or unsubstituted C₂₋C₁₆alkylene, substituted or unsubstituted C₂-C₁₆alkenylene, substituted or unsubstituted C₁-C₁₂alkyl(arylene), or substituted or unsubstituted aryl(C₁-C₁₂alkylene); and

R⁶ and R⁷ are independently hydrogen, oxygen, or C₁-C₄ alkyl; and hydrates and solvates thereof as particularly efficacious for the oral delivery of active agents, such as calcitonins, e.g. salmon calcitonin.

A calcitonin, e.g. salmon calcitonin, may be administered as the sole active ingredient or in conjunction with, e.g. as an adjuvant to, another therapeutic agent (Other Agent). Examples of Other Agents include, but are not limited to, agents useful for treating or preventing a bone-resorbing disease, a neoplastic disease, arthritis, a disease exacerbated by the presence of a high calcitonin, e.g. salmon calcitonin, activity or a disease improved by the presence of a calcitonin, e.g. salmon calcitonin; activating the function of a calcitonin, e.g. salmon calcitonin, in a bone cell; inhibiting the function of calcitonin, e.g. salmon calcitonin, in a cancer cell; inhibiting the expression of calcitonin, e.g. salmon calcitonin,-in a cell; and inhibiting the growth of a neoplastic cell. The Other Agent can be administered before, after or concurrently with the calcitonin, e.g. salmon calcitonin. In these embodiments, the time at which the calcitonin, e.g. salmon calcitonin, exerts their therapeutic effect on the patient overlaps with the time at which the Other Agent exerts its therapeutic effect on the patient.

In one embodiment, the Other Agent is useful for the treatment or prevention of a bone-loss disease (e.g., osteoporosis). Other Agents useful for the treatment or prevention of a bone-loss disease include, but are not limited to other calcitonins, (Asu-1,7) eel or human calcitonin, bisphosphonates (e.g., eitodronate, pamidronate, alendronate, risedronate, zoledronic acid, ibandronate, clodronate or tiludronate), Selective Estrogen Receptor Modulators (SERMs), such as tamoxifen, raloxifene, medroxyprogesterone, danizol and gestrinone, parathryoid hormone (“PTH”) or fragments or analogs thereof, compounds that release endogenous PTH (e.g., a PTH releasing compounds) and calcitonin fragments or analogs thereof.

In another embodiment, the Other Agent is useful for the treatment or prevention of a neoplastic disease. In one embodiment, the other therapeutic agent is useful for the treatment or prevention of cancer (e.g., cancer of the breast, ovary, uterine, prostate or hypothalamus). Other therapeutic agents useful for the treatment or prevention of cancer or a neoplastic disease include, but are not limited to, alkylating agents (e.g., nitrosoureas), an anti-metabolite (e.g., methotrexate or hydroxyurea), etoposides, campathecins, bleomycin, doxorubicin, daunorubicin, colchicine, irinotecan, camptothecin, cyclophosphamide, 5-fluorouracil, cisplatinum, carboplatin, methotrexate, trimetrexate, erbitux, thalidomide, taxol, a vinca alkaloid (e.g., vinblastine or vincristine) or a microtubule stabilizer (e.g., an epothilone).

Further illustrative examples of Other Agents useful for the treatment or prevention of cancer include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; ImiDs; interleukin II (including recombinant interleukin II, or rIL2), interferon-2a; interferon alpha-2b; interferon alpha-n1; interferon alpha-n3; interferon beta-I a; interferon gamma-I b; iproplatin; irinotecan hydrochloride; lanre6tide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; pellomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; SelCid; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; temozolomide; temodar; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other Agents useful for the treatment or prevention of cancer include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzopyranones, benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); cell-cycle inhibitors (e.g., flavopiridol A, tryprostatin B, p19ink4D); cyclin-dependent kinase inhibitors (e.g., roscovitine, olomucine and purine analogs); MAP kinase inhibitors (CNI-1493); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; lobenguane; iododoxorubicin; ipomeanol, 4-; iroplact; irsogladine; Isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzanlides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplafin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; retinoic acid (e.g., 9-cis RA); histone deacetylase inhibitors (e.g., sodium butyrate, suberoylanilide hydroxamic acid); TRAIL; ras famesyl protein transferase inhibitors; ras Inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustne; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsenfin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin.

In accordance with the foregoing the present invention provides in a yet further aspect:

A method as defined above comprising co-administration, e.g. concomitantly or in sequence, of a therapeutically effective amount of a calcitonin, e.g. salmon calcitonin, and at least one second drug substance, said second drug substance being a therapeutic agent against bone loss diseases, e.g. as indicated above.

Or, a therapeutic combination, e.g. a kit (=packaging), comprising of a therapeutically effective amount of a) a calcitonin, e.g. salmon calcitonin, and b) at least one second substance selected from a therapeutic agent against bone loss diseases, e.g. as indicated above. The kit may comprise instructions for its administration, e.g. may comprise an instruction stating that the salmon calcitonin should be taken in an amount between 0.1 mg to 2.5 mg of calcitonin, e.g. salmon calcitonin, preferably between 0.4 mg to 1.0 mg of calcitonin, e.g. salmon calcitonin.

Where the calcitonin, e.g. salmon calcitonin, is administered in conjunction with other therapeutic agents against bone loss diseases, dosages of the co-administered combination compound will of course vary depending on the type of co-drug employed, e.g. whether it is a bisphosphonate, a SERMs, a calcitonin, a PTH, a PTH fragment or a PTH analogue or others, on the specific drug employed, on the condition being treated and so forth. Pharmaceutical compositions comprising calcitonin, e.g. salmon calcitonin, and a second drug substance may be manufactured in conventional manner. A composition according to the invention may be administered by any conventional route, for example parenterally, e.g. in the form of injectable solutions (e.g. for zoledronic acid) or suspensions, or enterally, preferably orally (e.g. for Compound A, see above), e.g. in tablets or capsules.

The term “effective amount” In connection with a calcitonin, e.g. salmon calcitonin, means an. amount capable of treating a bone loss disease, in particular severe bone loss diseases, preferably severe osteoporosis, preferably severe osteoporosis in postmenopausal women, a neoplastic disease, arthritis, a disease exacerbated by the presence of cathepsin K activity or a disease improved by the presence of cathepsin K inhibitors; activating the function of cathepsin K in a bone cell; inhibiting the function of cathepsin K in a cancer cell; inhibiting the expression of cathepsin K in a cell; or inhibiting the growth of a neoplastic cell.

The term “effective amount” in connection with another therapeutic agent means an amount capable of treating or preventing a bone loss disease, in particular severe bone loss diseases, preferably severe osteoporosis, preferably severe osteoporosis in postmenopausal women, a neoplastic disease, arthritis, a disease exacerbated by the presence of estrogen or a disease improved by the presence of a calcitonin, e.g. salmon calcitonin; activating the function of calcitonin, e.g. salmon calcitonin, in a bone cell; inhibiting the function of calcitonin, e.g. salmon calcitonin, in a cancer cell; inhibiting the expression of calcitonin, e.g. salmon calcitonin, in a cell; or inhibiting the growth of a neoplastic cell, while the calcitonin, e.g. salmon calcitonin, is exerting its therapeutic or prophylactic effect.

The term “a severe form of bone loss diseases” means one severe form of bone loss diseases as defined above or can mean several severe forms of bone loss diseases.

The term “severe osteoporosis” is to be understood according to WHO, i.e. severe osteoporosis is considered to be present when the value for bone mineral content is more than 2.5 SDs below the mean for young adults and there is at least one so-called fragility fracture (a fracture assumed to be associated with osteoporosis because it occurred as a result of slight trauma).

The term “bone-mineral density” or BMD means that the amount of mineral in a specific area of bone is measured. The more mineral, the denser the bone. Mineral is measured in grams; area is measured in square centimeters—and BMD is described as grams per square centimeter.

The term “T-score” compares the bone density with that of the average healthy young adult woman at the age of 35. T-scores are based on a statistical measure called the standard deviation (SD), which reflects differences from the average score.

A “patient” is an animal, including, but not limited to, an animal such as a mammal including a human, e.g. a cow, monkey, horse, sheep, pig, chicken, turkey, quail, cat, dog, mouse, rat, rabbit, and guinea pig, preferably a human.

The invention is further described by way of illustration in the following Examples.

### EXAMPLE

### Example 1

### Serum CTX-I And Oral Salmon Calcitonin In Males

Serum CTX-I, or CrossLaps® (Nordic Bioscience Diagnostics A/S, cat. no. 4CRL4000) is an enzyme-immunoassay for quantitative assessment of bone resorption, and has been cleared by the FDA. It is based on two highly specific monoclonal antibodies against the amino acid sequence of EKAHD-β-GGR originating from the C-telopeptide of type I collagen. The aspartic acid residue (D) is β-isomerized. Standards, control, or unknown serum samples are pipetted into the appropriate microtitre wells coated with streptavidin, followed by application of a mixture of a biotinylated antibody and a peroxidase-conjugated antibody. Then, a complex between the CTX antigens, biotinylated antibody and peroxidase-conjugated antibody is generated, and this complex binds to the streptavidin surface via the biotinylated antibody. Following the one-step incubation at room temperature, the wells are emptied and washed. A chromogenic substrate is added and the colour reaction is stopped with sulfuric acid. Finally, the absorbance is measured.

The effect of oral salmon calcitonin (SCT) on the serum levels of CTX-I is investigated by randomising study participants (eight male volunteers) to receive three. single doses of SCT, a verum intravenous infusion of SCT or placebo in a five-period cross-over design (the study is described in detail in Buchlin et al., J Bone Miner Res., (2002); 17:1478-1485).

Serum CTX-I demonstrated a marked dose-dependent inhibition of bone resorption after oral and iv SCT, reaching a nadir 2-4 hours post-treatment and gradually returning to pre-treatment levels after 24 hours.

### Example 2

### Serum CTX-I/N-MID Osteocalcin And Oral Calcitonin In Postmenopausal Women

The N-MID® Osteocalcin ELISA (Nordic Bioscience Diagnostics A/S, cat. no. 3OSC4000) is an enzyme innuosorbent assay for quantitative assessment of bone formation, and has been cleared by the FDA. It is based upon the application of two highly specific monoclonal antibodies (Mabs) against human osteocalcin. An antibody recognizing the midregion (amino acids 20-29) is used as the capture antibody and for detection a peroxidase conjugated antibody recognizing the N-terminal region (amino acids 10-16) is used. In addition to intact osteocalcin (amino acid 1-49) the N-terminal-Mid fragment (amino acids 1-43) is also detected. Standards, control and unknown samples are pipetted into the appropriate microtitre wells coated with streptavidin. Then a mixture of a blotinylated antibody and a peroxidase conjugated antibody is added. Following incubation for 2 hours at room temperature the wells are washed and a chromogenic substrate is added and the colour reaction is stopped with sulfuric acid. Finally, the absorbance is measured.

The response of serum CTX-I and N-MID® Osteocalcin to SCT in postmenopausal women is examined in a multi-center, randomised, double-blind, placebo-controlled, dose-ranking clinical trial including 277 study participants aged 55-85 years (the study has been described in detail in Tank\acute{0} et al., J Bone Miner Res., in print). The study participants receives active treatment (daily doses of 0.15, 0.4, 1.0, or 2.5 mg or 1.0 mg every other day) or placebo for three months and serum CTX-I as well as N-MID® Osteocalcin are measured frequently during a 24-hours period before drug administration and after one and three months.

After the first dose, serum CTX-I decreases dose-dependently (−60.8 to −81.8% from baseline) compared with placebo. However, the changes of N-MID® Osteocalcin remaines non-significant. These data suggest that SCT suppresses bone resorption while maintaining bone formation.

1. A method for the treatment of a severe form of bone loss diseases in a patient in need of such treatment which comprises administering an effective amount of a calcitonin to the patient.

2. (canceled)

3. A pharmaceutical composition which incorporates as an active agent a calcitonin, for use in the treatment of a severe form of bone loss diseases.

4. A method according to claim 1, wherein the disease is a severe form of osteoporosis.

5. A method according to claim 1, wherein the disease is severe osteoporosis in postmenopausal women.

6. The pharmaceutical composition according to claim 3, said composition comprising less than 2.5 mg salmon calcitonin.

7. The pharmaceutical composition according to claim 6, wherein said composition comprising between 0.4 mg and 1.0 mg of salmon calcitonin.

8. (canceled)

9. A method for the treatment of osteoporosis in a patient in need of such a treatment which comprises administering less than 2.5 mg of salmon calcitonin to the patient.

10. A method for the treatment of severe osteoporosis in a patient in need of such a treatment which comprises administering less than 2.5 mg of salmon calcitonin to the patient.
